(19) United States
(12) Patent Application Publication
(10) Pub. No.: US 2010/0010570 A1
(43) Pub. Date: Jan. 14, 2010

(54) AUDITORY PROSTHESIS

(76) Inventors: David Bruce Grayden, Victoria (AU); Graeme Milbourne Clark, Victoria (AU)

Correspondence Address: FROMMER LAWRENCE & HAUG, 745 FIFTH AVENUE- 10TH FL., NEW YORK, NY, 10151, US

(21) Appl. No.: 12/375,052

(22) PCT Filed: Jul. 26, 2007
(86) PCT No.: PCT/AU07/01043

§ 371 Date: Sep. 25, 2009

(30) Foreign Application Priority Data
Jul. 26, 2006 (AU) 2006904062

(51) Int. Cl. A61F 11/04; A61N 1/36; A61N 1/05

(52) U.S. Cl. 607/57; 607/137

(57) ABSTRACT

An auditory prosthesis (100) comprising, at least one audio transducer (110) for receiving sound and producing at least one audio signal based on the received sound, processing circuitry (170) configured to process the audio signal to output electrophonic stimuli, and at least one first electrode (264) electrically connected to the processing circuitry for applying the electrophonic stimuli to a cochlea of a user of the auditory prosthesis.

### FIELD

The present invention relates to an auditory prosthesis.

### BACKGROUND TO THE INVENTION

The multi-channel cochlear implant has now been accepted as effective for providing speech understanding to people with sensorineural hearing loss. Speech perception outcomes have been so beneficial that the criteria for cochlear implant candidacy have extended to include people with a severe degree of hearing loss. Therefore, an increasing number of cochlear implant recipients have residual hearing in the implanted ear, particularly in the low frequency regions. In order to better preserve this low frequency hearing, surgical techniques and shorter electrode arrays have been employed that only provide electroneural stimulation of auditory nerves outside this low frequency range. This allows the simultaneous application, in the same ear, of electrical stimulation of the auditory nerve using the cochlear implant, and acoustic stimulation of the residual hearing using an external hearing aid.

There is a need for an alternative technique suitable for cases where an implant recipient has some residual hearing.

### SUMMARY OF THE INVENTION

In a first aspect the invention provides an auditory prosthesis comprising:
at least one audio transducer for receiving sound and producing at least one audio signal based on the received sound;
processing circuitry configured to process the audio signal to output electrophonic stimuli; and
at least one first electrode electrically connected to the processing circuitry for applying the electrophonic stimuli to a cochlea of a user of the auditory prosthesis.

In an embodiment the processing circuitry is configured to process the audio signal to output electroneural stimuli and the auditory prosthesis further comprises at least one second electrode for applying the electroneural stimuli to the cochlea of the user.

In an embodiment the at least one first electrode and the at least one second electrode form an electrode array adapted to be inserted into the cochlea of a user.

In an embodiment each first electrode is arranged on the array so as to be located apically of each second electrode.

In an embodiment the processing circuitry comprises an external processor and an internal stimulator that provides both electrophonic and electroneural stimulation.

In an embodiment the processing circuitry comprises:
an external processor;
a first internal stimulator for outputting electrophonic stimuli to each at least one first electrode; and
a second internal stimulator for outputting electroneural stimuli.

In an embodiment the processing circuitry comprises:
a first external processor and a first internal stimulator for outputting electrophonic stimuli to each first electrode; and
a second external processor and a second internal stimulator for outputting electroneural stimuli to each second electrode.

In an embodiment the processing circuitry is configured such that the electrophonic stimuli correspond to portions of the audio signal in a first frequency range and the electroneural stimuli correspond to portions of the audio signal in a second frequency range.

In an embodiment the processing circuitry is configured to allow adjustment of the first frequency range to include frequencies of sound for which the user has residual hearing.

In an embodiment the processing circuitry is configured to allow adjustment of the second frequency range to include frequencies of sound for which the user has profound or severe hearing loss.

In an embodiment the at least one first electrode is adapted to apply stimulation to a region of the cochlea with little or no residual hearing to thereby apply electrophonic stimulation.

In an embodiment the processing circuitry is configured to output electrophonic and electroneural stimuli to each first and second electrode at a stimulation rate having a frequency greater than the estimated highest frequency of residual hearing.

In an embodiment the stimulation rate for electrophonic stimuli is equal to or greater than twice the estimated highest frequency of residual hearing.

In an embodiment the electrophonic stimuli are amplitude modulated.

In a second aspect, the invention provides processing circuitry for an auditory prosthesis, the processing circuitry arranged to receive an audio signal as an input and to process the audio signal to output electrophonic stimuli in a form such that, in use, the electrophonic stimuli may be applied by at least one first electrode to a cochlea of a user.

In an embodiment the Processing circuitry is further configured to process the audio signal to output electroneural stimuli that may be applied by at least one second electrode to the cochlea of the user.

In an embodiment the processing circuitry comprises an external processor and an internal stimulator that provides both electrophonic and electroneural stimulation.

In an embodiment the processing circuitry comprises:
an external processor;
a first internal stimulator for outputting electrophonic stimuli to each at least one first electrode; and
a second internal stimulator for outputting electroneural stimuli.

In an embodiment the processing circuitry comprises:
a first external processor and a first internal stimulator for outputting electrophonic stimuli to each first electrode; and
a second external processor and a second internal stimulator for outputting electroneural stimuli to each second electrode.

In an embodiment the processing circuitry is configured such that the electrophonic stimuli correspond to portions of the audio signal in a first frequency range and the electroneural stimuli correspond to portions of the audio signal in a second frequency range.

In an embodiment the processing circuitry is configured to allow adjustment of the first frequency range to include frequencies of sound for which the user has residual hearing.

In an embodiment, the processing circuitry is configured to allow adjustment of the second frequency range to include frequencies of sound for which the user has profound or severe hearing loss.

In an embodiment, the processing circuitry is configured to output electrophonic and electroneural stimuli to each first and second electrode at a stimulation rate having a frequency greater than the estimated highest frequency of residual hearing.

In an embodiment, the stimulation rate for electrophonic stimuli is equal to or greater than twice the estimated highest frequency of residual hearing.

In an embodiment, the electrophonic stimuli are amplitude modulated.

In a third aspect, the invention provides an auditory prosthesis electrode comprising at least one first electrode adapted to apply electrophonic stimuli to a cochlea of a user.

In an embodiment, the at least one first electrode is at least one electrode of an electrode array further comprising at least one second electrode adapted to apply electroneural stimulation to the cochlea of the user.

In an embodiment, each first electrode is arranged on the array so as to be located apically of each second electrode when implanted in the user.

In an embodiment, the at least one first electrode is adapted to apply stimulation to a region of the cochlea with little or no residual hearing to thereby apply electrophonic stimulation.

In a fourth aspect, the invention provides a method of assisting hearing in a hearing impaired user comprising applying electrophonic stimuli to a cochlea of the user via at least one first auditory prosthesis electrode implanted in the user.

In an embodiment, the method comprises applying stimulation to a region of the cochlea with little or no residual hearing to thereby apply the electrophonic stimuli.

In an embodiment, the method comprises placing the at least one first auditory prosthesis electrode close to the basilar membrane of the cochlea.

In an embodiment, the method comprises applying the electrophonic stimuli at a stimulation rate above the frequency of the user's residual hearing.

In an embodiment, the method comprises applying the electrophonic stimuli a stimulation rate equal to or greater than twice the estimated highest frequency of residual hearing.

In an embodiment, the method comprises applying electroneural stimuli via at least one second auditory prosthesis electrode.

### BRIEF DESCRIPTION OF THE DRAWINGS

Exemplary embodiments of the invention will now be described with reference to the accompanying drawings in which:

FIG. 1 is a block diagram of an auditory prosthesis of a first embodiment;

FIG. 2 is a block diagram of an auditory prosthesis of a second embodiment;

FIG. 3 is a block diagram of an auditory prosthesis of a third embodiment;

FIG. 4 is a block diagram of an auditory prosthesis of a fourth embodiment;

FIG. 5 is a diagrammatic representation of an auditory prosthesis installed in the ear of a user;

FIG. 6 is a block diagram of the functions performed by the auditory prosthesis of FIG. 1;

FIG. 7 is a block diagram of the functions performed by the auditory prosthesis of FIG. 2;

FIG. 8 shows an example of stimuli applied by the auditory prosthesis of FIG. 2;

FIG. 9 is an alternative example of stimuli applied by the auditory prosthesis of FIG. 2; and

FIG. 10 is an alternative example of stimuli applied by the auditory prosthesis of FIG. 2.

### DESCRIPTION OF THE PREFERRED EMBODIMENT

The preferred embodiment takes advantage of the fact that low frequency hearing is available through the basilar membrane vibration and excitation of hair cells for many potential or current cochlear implant users. The preferred embodiment employs electrical stimulation of the cochlea, preferably in the region of the basilar membrane to provide electrophonic hearing. Electrophonic stimulation can be employed in addition to electroneural stimulation.

Electrophonic hearing is a perception of sound that occurs when electrical stimulation of the cochlea results in mechanical stimulation of hair cells through vibration of the basilar membrane. This is as opposed to electroneural hearing that results from direct electrical stimulation of the auditory nerve, bypassing the hair cells.

Low frequency electrophonic hearing bypasses the middle ear and most basal part of the cochlea. Based on the experience of the inventors, this will deliver higher fidelity sound than electroneural stimulation alone by providing improved representation of the harmonics of low-frequency sound and by improved entrainment of neural responses, which will aid music appreciation and allow better perception of lower formants and fundamental frequency of speech. In addition, the electrophonic stimulation may be applied to stimulate the basilar membrane apical to the location of the electrode or electrodes thus any tissue growth, including new bone, in the base of the cochlea will not impair the travelling wave. Such tissue growth may result from the insertion of the prosthesis.

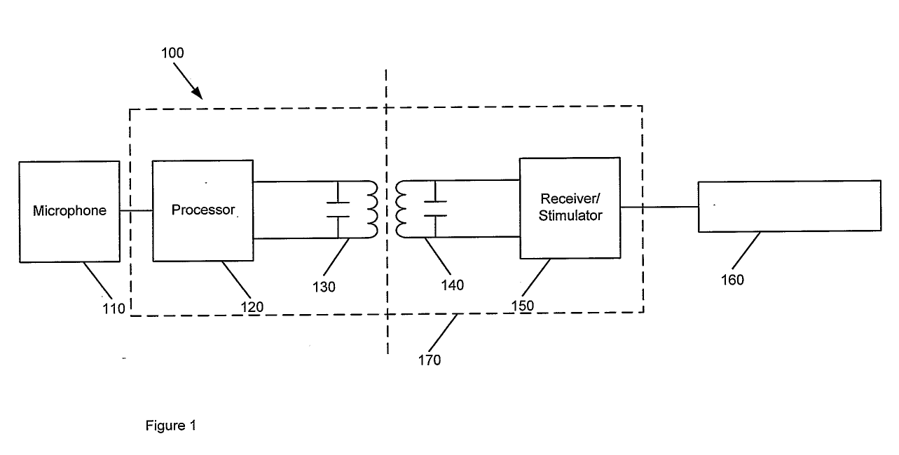

FIG. 1 illustrates an auditory prosthesis 100 of a first embodiment which is suitable to be used, for example, in a user with some residual hearing and who has previously had implanted an auditory prosthesis in such a manner that low frequency hearing has been preserved.

The auditory prosthesis 100 comprises a microphone 110 which produces an audio signal that is fed to a processor 120 and subjected to auditory processing. The output of the processor 120 is transmitted via an external coil 130 and an internal coil 140 to a receiver/stimulator 150 that outputs electrophonic stimuli to an electrophonic electrode 160 to apply the electrophonic stimuli to the user. Accordingly, the processor, 120, the coils, 130,140 and the receiver stimulator 150, collectively provide processing circuitry 170 for the auditory prosthesis 100.

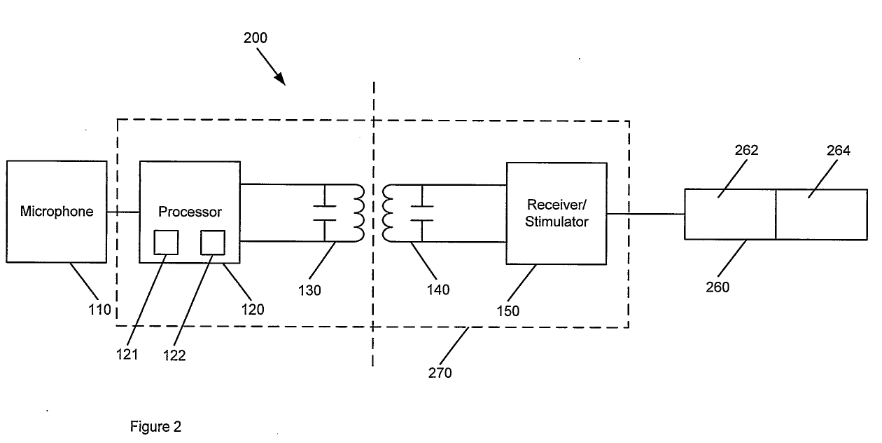

The embodiment of FIG. 2 provides an auditory prosthesis for generating electrical stimuli for application to a cochlea via auditory prosthesis electrodes that generates electroneural stimulation for frequencies of sound for which the user has severe or profound hearing loss and electrophonic stimulation for frequencies of sound for which the user has residual hearing.

Electrophonic stimulation using sinusoidal current stimulation of the cochlea causes vibration and hair cell response at the position of the electrode in the cochlea and at the characteristic frequency corresponding to the frequency of the sinusoidal current. The stimulating electrode initiates a travelling wave on the basilar membrane that propagates in a manner similar to the normal basilar membrane travelling wave to its appropriate position on the basilar membrane.

In a manner similar to sinusoidal stimulation, pulsatile stimulation excites the characteristic position on the basilar membrane corresponding to the frequency of the stimulus.

There are a number of methods that may be employed to deliver the combined electroneural and electrophonic stimuli. Herein we disclose one strategy for delivering electroneural stimuli and a number of strategies for delivering electrophonic stimuli. There are two classes of stimulation that may be used for electrophonic stimulation of residual hearing: amplitude modulation of pulsatile current stimuli and analogue stimulation (or a piece-wise analogue stimulation strategy).

FIG. 2 illustrates an auditory prosthesis designed to deliver both electroneural and electrophonic stimulation. The audio signal from microphone 110 is processed by processing circuitry 270 which has a processor 120, external coil 130, internal coil 140 and receiver/stimulator 150.

In this embodiment, processing circuit 270 outputs both electroneural stimuli and electrophonic stimuli to the electroneural electrodes 262 and the electrophonic electrodes 264 of electrode array 260. The processor 120 of processing circuit 270 incorporates an electroneural processing portion 121 for implementing elements 630,640 and 650 of FIG. 7 and an electrophonic processing portion 122 for implementing elements 635 and 655 of FIG. 7 which are described in further detail below.

In this embodiment, the implanted array of electrodes contains a set of electrodes that are nominated for electroneural stimulation (“S2” electrodes), for example, six electrodes (or electrode pairs for bipolar stimulation such as High-Focus electrodes) and an electrode or set of electrodes that are nominated for electrophonic stimulation (the “S1” electrodes). The S1 stimulation may be created between more than one electrode such as an electrode pair or even more electrodes for focussing current.

The S2 electrodes are distributed along an array such that they lie around the basal turn of the cochlea in the same way as existing cochlear implants, although there will most likely be fewer electrodes (N2 electrodes), such as a short array. These electrodes are assigned frequency bands to represent. The electrodes are stimulated in using any appropriate cochlear implant sound processing strategy. However, the frequency bands do not represent the entire spectrum as with current cochlear implant frequency allocation schemes. Instead they cover a frequency range that starts at a point above the highest usable residual hearing frequency available to a user (hereafter referred to as RH) up to the maximum permitted by the stimulation strategy (for example around 8000 Hz).

This embodiment employs a CIS-like stimulation scheme, where the electrodes are stimulated at a fixed rate (S) which is at least twice the rate of RH. Thus if RH is 500 Hz, then the rate of stimulation for the electrodes is at least 1000 pulses per second per electrode (pps/electrode) ensuring that the rate of stimulation is well above the highest residual hearing level of the user; i.e., S>=1000 pps/electrode, for example, S=2000 pps/electrode. However, the total stimulation rate (TS) is S*(N2+1) to ensure that there is a ‘spare’ stimulation ‘slot’ available for the S1 electrode(s) in each cycle (TS=18000 pps for our example). Persons skilled in the art will appreciate that the S1 electrode could be stimulated at a different rate to the S2 electrodes, for example, once every second cycle or twice a cycle. In other embodiments discussed below where the S1 and S2 electrodes are provided separately (i.e. not as part of the same electrode array), the S1 stimulation rate may be independent of the S2 stimulation rate.

The S1 electrode is stimulated in the spare stimulation slot for each stimulation cycle. The incoming sound frequency is filtered between two frequencies (between RL and RH) within the usable residual hearing region of the user. RL is set to a low end of the residual hearing at some frequency at or above 0 Hz (for example, 50 Hz). RH is set to the highest usable residual hearing frequency (500 Hz in our example). The stimulation of the S1 electrode is at a level that depends on the output of the RL-RH band-pass filter. Thus, the S1 electrode will carry a stimulation sequence of monophasic pulses that are amplitude modulated by the output of the band-pass filter. Based on the inventors' experience, the result should be that the basilar membrane at the position of the S1 electrode will vibrate at the S frequency (2000 pps in this example) and will also vibrate with a complex pattern resulting from the amplitude modulation. The S frequency vibration will propagate along the basilar membrane as a travelling wave to the position best frequency position for S Hz. This should not cause any hearing sensation because the patient will not have residual hearing at this frequency. The complex vibration pattern resulting from the amplitude modulation will propagate along the Basilar Membrane 3 to its component frequency's best positions, which are within the useful range of residual hearing and so will be perceived by the user as the correct frequencies.

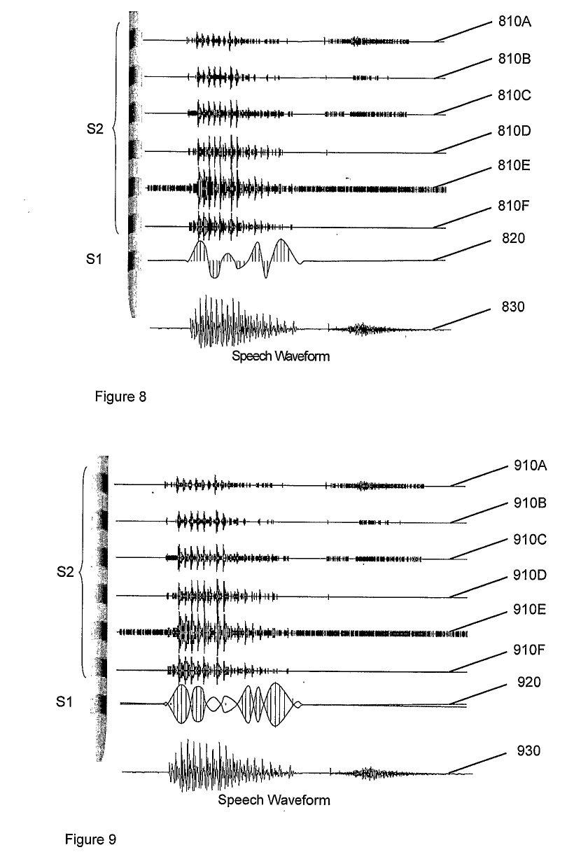

FIG. 8 provides an illustrative example of this strategy. The input is the sound wave form 830. Six electrodes are part of S2 and are stimulated with biphasic pulses for the higher-frequency sounds producing 810 output 810A-810F. The seventh electrode is S2 and it is stimulated using monophasic pulses whose amplitude are controlled by the output of the band-pass filter for the lower-frequency sounds. Note that the rate of stimulation shown 820 for S1 is much lower than S2 for illustrative purposes—in practice it should be the same rate as S2.

FIG. 9 illustrates an alternative strategy with input sound wave form 930 where, the pulsatile stimuli 920 on the S1 electrode may be biphasic pulses at the same or different (possibly a multiple of) rate of stimulation 910 as the S2 electrodes as shown in FIG. 9. Otherwise the strategy is the same as that given above.

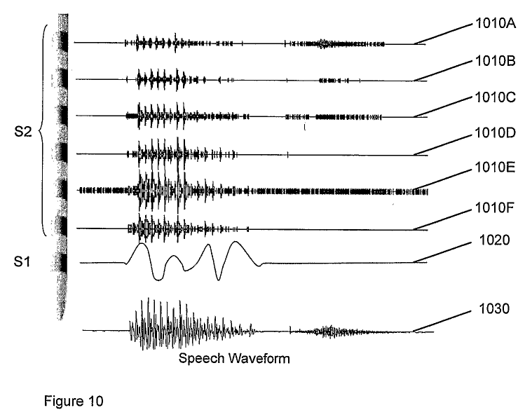

Alternatively, analogue stimulation 1020 may be used for the S1 electrode as illustrated in FIG. 10 for input sound wave form 1030. A specific electrode or multiple electrodes are stimulated using analogue stimulation, essentially producing stimulation that replicates the output of a low-pass or band-pass filter. This would be a different stimulation scheme on the electrode or electrodes providing electrophonic stimulation than the other electrodes in the array that perform electroneural stimulation 1010.

In a further alternative strategy, piece-wise analogue stimulation may be used for the S1 electrode. This is a quantised approximation to an analogue stimulation method where stimulation on an electrode or electrodes is updated at some regular or irregular interval but maintains a constant current between updates. The benefit of this method is that charge delivery can be carefully measured and updates can occur at times that current pulses on electroneural stimulating electrodes are not being made. The result of this stimulation is much like that shown in FIG. 10 except that rather than a smooth waveform, there will be steps where each pulse holds its current level and then the next pulse either increases or decreases the level.

Monophasic pulsatile stimulation is preferable as the spectral shape will be maintained for the low-frequency sounds. However it may be possible to employ biphasic stimulation.

It is preferred that the monophasic/biphasic stimuli should be as short duration as possible while delivering sufficient current in order to minimise the amount of charge delivered with each pulse and to make the pulses appear as close as possible to impulses. The former feature will reduce the amount of electroneural stimulation and also reduce the amount of charge delivered that needs to be balanced by pulses of opposite polarity. The latter feature will maintain the spectrum of the low frequency sounds closer to its original form.

In the above embodiments the pulsatile stimulation rate or piece-wise analogue stimulation update rate are at least two times the high-pass cut-off of the amplitude extraction filter in order to satisfy the Nyquist criterion. The rate is also above the highest useful residual hearing frequency available to avoid perception of the stimulation rate.

The sound is band-pass filtered at low frequencies. The low end is chosen at a high enough frequency to ensure that charge balance is maintained over a short enough period, say 50 Hz. The high frequency cut-off should be placed at the frequency wherein sufficient hearing is available to be excited by electrophonic stimulation. This latter frequency would most likely be around 500 Hz but could be higher depending on the subject.

As illustrated in FIG. 5, a short electrode array 260 is placed in the basal turn in the cochlea for electrical stimulation of the auditory nerve in the manner currently performed for cochlear implants. FIG. 5 shows an external processor 120 mounted behind the ear 1 of a user. Microphone 110 receives ambient sound, this is processed by the processor 120 in order to drive external coil 130. Internal coil 140 picks up the signal transmitted by the external coil 130 and receiver/stimulator 150 generates both electroneural and electrophonic stimuli for transmission to the electrode array 260.

The electrophonic stimulation electrode or electrodes 264 are placed in order to focus current at the site of the electrode or electrodes. The electrode 264 is placed in a region of the cochlea with low residual hearing level at a higher characteristic frequency than the highest feasible level of residual hearing. The electrodes 264 are placed close to the basilar membrane 3 to maximise the instigation of the travelling wave. The electrodes are designed and placed to minimise current interactions with the electrodes 262 that stimulate electroneural hearing.

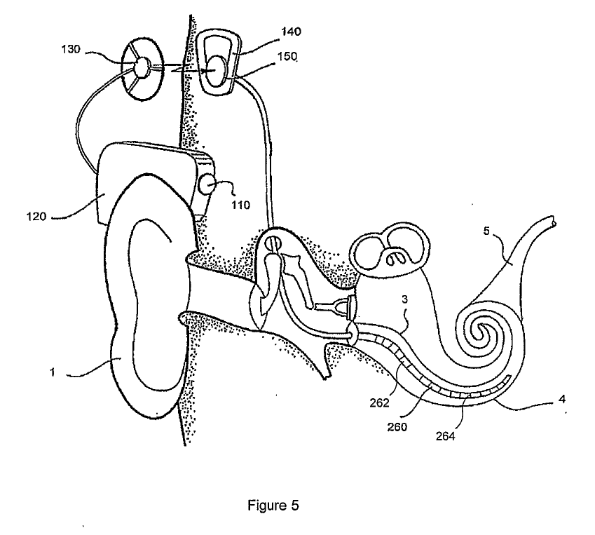

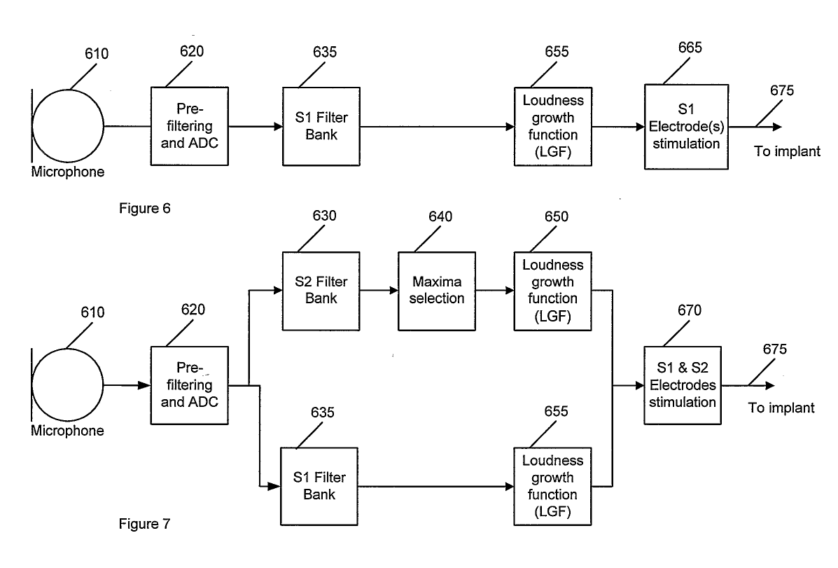

Signal processing schemes for the embodiments of FIGS. 1 and 2 respectively are illustrated in FIGS. 6 and 7.

Referring to FIG. 6, Microphone 610 receives auditory input which is filtered and converted to a digital signal by the pre-filtering and analogue to digital converter 620. The S1 Filter Bank 635 (typically but not necessarily, a single filter) filters the signal into frequency bands. A Loudness growth function 655 determines the appropriate level of excitation. Stimulation control signals 665 are then passed in an appropriate manner to the implanted S1 electrode or electrodes 675.

Microphone 610 receives auditory input which is filtered and converted to a digital signal by the Pre-filtering and ADC 620. The S2 Filter Bank 630 filters the signal into frequency bands, one band for each S1 electrode. Maxima selection 640 chooses which electrodes to stimulate and a Loudness growth function 650 determines the appropriate level of excitation. The S1 Filter Bank 635 (typically, but not necessarily, a single filter) filters the signal into frequency bands. A Loudness growth function 655 determines the appropriate level of excitation. Stimulation control signals 670 are then passed in an appropriate manner to the implanted S1 and S2 electrodes 675.

Persons skilled in the art will appreciate there may be variations, for example depending on the embodiment, the electrophonic current delivery may be:
The most apical electrode of the short array, stimulated in monopolar mode in the same way as the other electrodes.
The two most apical electrodes of the short array, stimulated in bipolar mode to create more localised currents or in monopolar modes in a way that focuses current to the local area.
An extra electrode or electrodes placed on the end of the short array but further along than normal to further displace it from the other electrodes.
An extra electrode or electrodes placed separately to the short array. For example, on the wall of the cochlea or some other location, preferably away from the short array.

In addition, a ground electrode may be placed on the outer wall of the cochlea or some other location where it creates a path that allows conduction of current from the electrophonic electrode or electrodes away from the region of excitation of the electroneural stimulating electrodes.

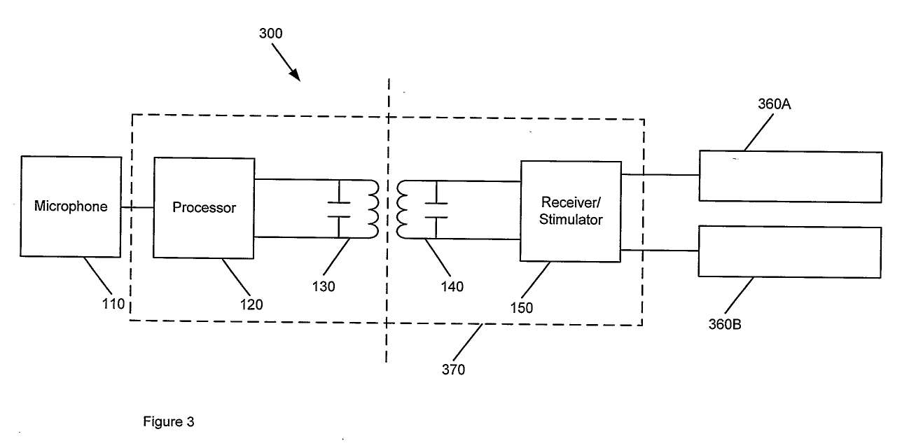

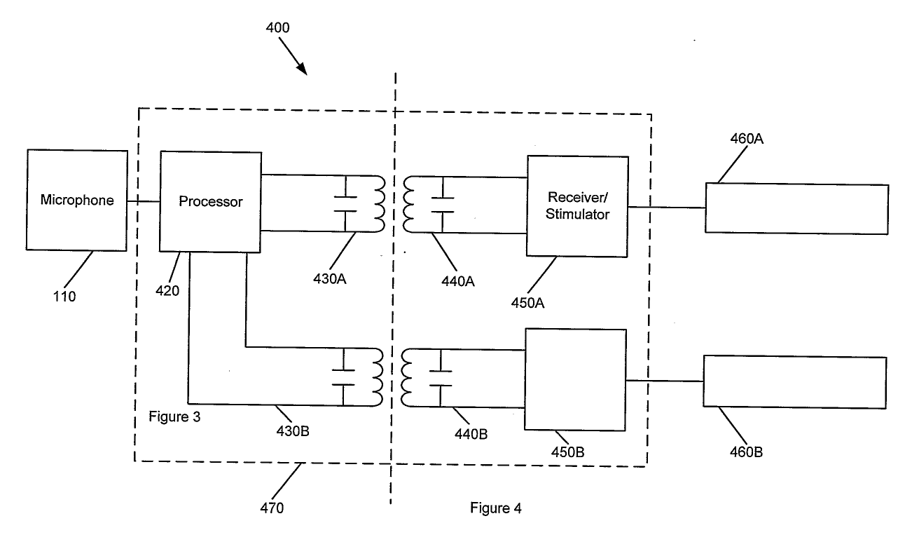

Other embodiments are possible, for example the auditory prosthesis of FIG. 3 is substantially the same as that illustrated in FIG. 2, except in this embodiment separate electrodes are provided for the electroneural and the electrophonic stimulation. That is, the processing circuitry 370 outputs electroneural stimuli to electrode array 360A and outputs electrophonic stimuli to electrode 360B.

FIG. 4 illustrates a further variant. In this case, the implantable portions of the auditory prosthesis are provided by separate circuits, a first implantable circuit has a first internal coil, a first receiver/stimulator 450A and an electrode array 460A for applying electroneural stimuli to the cochlea. A second internal implant has a second internal coil 440B, second internal receiver/stimulator 450B and an electrophonic electrode 460B. In order to drive the internal coils 440, separate external coils 430A,430B are provided both of which receive output signals from processor 420. This configuration may be suitable for retrofitting an electrophonic electrode to a user who has a cochlear implant and some residual hearing. Employing a single processor externally keeps the equipment to a manageable size but a pair of processors could be used to parallel process the output of a single audio transducer or separate audio transducers.

While the above embodiments employ an external processor, the processor may be deployed internally in a totally implantable auditory prosthesis.

Further, the preferred implementation is based on Continuous Interleaved Sampling (CIS) or Spectral Maxima Sound Processor (SMSP) strategies that have fixed rates of stimulation and extract the envelope of the signal to control stimulus levels. Other strategies may also be used that do not have fixed rates of stimulation or set levels of stimulation based on other measures of the filter bank outputs. Examples of strategies that do not use fixed rates of stimulation include, but are not limited to, the Spike-based Temporal Auditory Representation (STAR) strategy described in AU2005237146 the disclosure of which is incorporated herein by reference, the Travelling Wave strategy described in US2003171786 the disclosure of which is incorporated herein by reference and the Peak-Derived Timing strategy (PDT) described in US2004172101 the disclosure of which is incorporated herein by reference. These strategies use aspects of the filter bank outputs to create sequences of electrical stimuli. Examples of strategies that use other measures of filter bank outputs include, but are not limited to, the strategies named above and the Specific Loudness (SPeL) strategy. These strategies use measures other than the filter bank envelopes, such as peak output level, to determine the levels of electrical stimuli.

Other variations will be apparent to persons skilled in the art and shall be understood as falling within the scope of the invention described herein.

1. An auditory prosthesis comprising: at least one audio transducer for receiving sound and producing at least one audio signal based on the received sound; processing circuitry configured to process the audio signal to output electrophonic stimuli; and at least one first electrode electrically connected to the processing circuitry for applying the electrophonic stimuli to a cochlea of a user of the auditory prosthesis.

2. An auditory prosthesis as claimed in claim 1 wherein the processing circuitry is configured to process the audio signal to output electroneural stimuli and the auditory prosthesis further comprises at least one second electrode for applying the electroneural stimuli to the cochlea of the user.

3. An auditory prosthesis as claimed in claim 2 wherein the at least one first electrode and the at least one second electrode form an electrode array adapted to be inserted into the cochlea of a user.

4. An auditory prosthesis as claimed in claim 3 wherein each first electrode is arranged on the array so as to be located apically of each second electrode.

5. An auditory prosthesis as claimed in claim 1 wherein the processing circuitry comprises an external processor and an internal stimulator that provides both electrophonic and electroneural stimulation.

6. An auditory prosthesis as claimed in claim 2, wherein the processing circuitry comprises:
an external processor;
a first internal stimulator for outputting electrophonic stimuli to each at least one first electrode; and
a second internal stimulator for outputting electroneural stimuli.

7. An auditory prosthesis as claimed in claim 2, wherein the processing circuitry comprises:
a first external processor and a first internal stimulator for outputting electrophonic stimuli to each first electrode; and
a second external processor and a second internal stimulator for outputting electroneural stimuli to each second electrode.

8. An auditory prosthesis as claimed in claim 2, wherein the processing circuitry is configured such that the electrophonic stimuli correspond to portions of the audio signal in a first frequency range and the electroneural stimuli correspond to portions of the audio signal in a second frequency range.

9. An auditory prosthesis as claimed in claim 8, wherein the processing circuitry is configured to allow adjustment of the first frequency range to include frequencies of sound for which the user has residual hearing.

10. An auditory prosthesis as claimed in claim 8, wherein the processing circuitry is configured to allow adjustment of the second frequency range to include frequencies of sound for which the user has profound or severe hearing loss.

11. An auditory prosthesis as claimed in claim 1, wherein the at least one first electrode is adapted to apply stimulation to a region of the cochlea with little or no residual hearing to thereby apply electrophonic stimulation.

12. An auditory prosthesis as claimed in claim 2, wherein the processing circuitry is configured to output electrophonic and electroneural stimuli to each first and second electrode at a stimulation rate having a frequency greater than the estimated highest frequency of residual hearing.

13. An auditory prosthesis as claimed in claim 12 wherein the stimulation rate for electrophonic stimuli is equal to or greater than twice the estimated highest frequency of residual hearing.

14. An auditory prosthesis as claimed in claim 1, wherein the electrophonic stimuli are amplitude modulated.

15. Processing circuitry for an auditory prosthesis, the processing circuitry arranged to receive an audio signal as an input and to process the audio signal to output electrophonic stimuli in a form such that, in use, the electrophonic stimuli may be applied by at least one first electrode to a cochlea of a user.

16. Processing circuitry as claimed in claim 15, further configured to process the audio signal to output electroneural stimuli that may be applied by at least one second electrode to the cochlea of the user.

17. Processing circuitry as claimed in claim 15 comprising an external processor and an internal stimulator that provides both electrophonic and electroneural stimulation.

18. Processing circuitry as claimed in claim 16, comprising:
an external processor;
a first internal stimulator for outputting electrophonic stimuli to each at least one first electrode; and
a second internal stimulator for outputting electroneural stimuli.

19. Processing circuitry as claimed in claim 16, comprising: a first external processor and a first internal stimulator for outputting electrophonic stimuli to each first electrode; and a second external processor and a second internal stimulator for outputting electroneural stimuli to each second electrode.

20. Processing circuitry as claimed in claim 16, configured such that the electrophonic stimuli correspond to portions of the audio signal in a first frequency range and the electroneural stimuli correspond to portions of the audio signal in a second frequency range.

21. Processing circuitry as claimed in claim 20 configured to allow adjustment of the first frequency range to include frequencies of sound for which the user has residual hearing.

22. Processing circuitry as claimed in claim 20 configured to allow adjustment of the second frequency range to include frequencies of sound for which the user has profound or severe hearing loss.

23. Processing circuitry as claimed in claim 16, configured to output electrophonic and electroneural stimuli to each first and second electrode at a stimulation rate having a frequency greater than the estimated highest frequency of residual hearing.

24. Processing circuitry as claimed in claim 23 wherein the stimulation rate for electrophonic stimuli is equal to or greater than twice the estimated highest frequency of residual hearing.

25. Processing circuitry as claimed in claim 15, wherein the electrophonic stimuli are amplitude modulated.

26. An auditory prosthesis electrode comprising at least one first electrode adapted to apply electrophonic stimuli to a cochlea of a user.

27. An auditory prosthesis electrode as claimed in claim 26, wherein the at least one first electrode is at least one electrode of an electrode array further comprising at least one second electrode adapted to apply electroneural stimulation to the cochlea of the user.

28. An auditory prosthesis electrode as claimed in claim 27, wherein each first electrode is arranged on the array so as to be located apically of each second electrode when implanted in the user.

29. An auditory prosthesis electrode as claimed in claim 26, wherein the at least one first electrode is adapted to apply stimulation to a region of the cochlea with little or no residual hearing to thereby apply electrophonic stimulation.

30. A method of assisting hearing in a hearing impaired user comprising applying electrophonic stimuli to a cochlea of the user via at least one first auditory prosthesis electrode implanted in the user.

31. A method as claimed in claim 30 comprising applying stimulation to a region of the cochlea with little or no residual hearing to thereby apply the electrophonic stimuli.

32. A method as claimed in claim 31 comprising placing the at least one first auditory prosthesis electrode close to the basilar membrane of the cochlea.

33. A method as claimed in claim 30 comprising applying the electrophonic stimuli at a stimulation rate above the frequency of the user's residual hearing.

34. A method as claimed in claim 33 comprising applying the electrophonic stimuli a stimulation rate equal to or greater than twice the estimated highest frequency of residual hearing.

35. A method as claimed in claim 30 comprising applying electroneural stimuli via at least one second auditory prosthesis electrode.
